(19) United States
(12) Patent Application Publication
(10) Pub. No.: US 2025/0153158 A1
(43) Pub. Date: May 15, 2025

(54) CATALYST COMPOSITION AND METHOD FOR PRODUCING A CATALYST

(71) Applicant: SHELL USA, INC., Houston, TX (US)

(72) Inventors: Josiane Marie-Rose GINESTRA, Richmond, TX (US); Cornelius Mark BOLINGER, Sugar Land, TX (US); Lynn Henry SLAUGH, Cypress, TX (US)

(21) Appl. No.: 18/835,469

(22) PCT Filed: Feb. 18, 2022
(86) PCT No.: PCT/US2022/016979

§ 371 Date: Aug. 2, 2024

(30) Foreign Application Priority Data
Feb. 18, 2022 (WO) PCT/US2022/016979

(51) Int. Cl. B01J 37/02; B01J 21/04; B01J 23/04; B01J 35/61

(52) U.S. Cl. CPC B01J 37/0201; B01J 21/04; B01J 23/04; B01J 35/615; B01J 35/617; B01J 35/618

(57) ABSTRACT

An isomerization catalyst composition includes an alumina based catalyst, wherein the alumina based catalyst has a pore volume in pores of less than 70 Å pore diameter of less than about 5% of Total Pore Volume, a pore volume in pores of greater than 350 Å pore diameter of less than 10% of Total Pore Volume, a median pore diameter by volume of less than 200 Å, a water pore volume of less than 1.2 cc/g and a surface area of greater than 130 m2/g. The isomerization catalyst composition may include Group I cations, Group II cations and mixtures thereof.

### BACKGROUND

Ethylene can be oligomerized to higher molecular weight linear monoolefins. A variety of suitable oligomerization catalysts and processes are known. Such olefins comprise for example, those of the C₄-C₁₀ range, useful as comonomers in plastics, e.g., LLDPE, or as synthetic lubricants; those of the C₁₂-C₂₀ range, useful as detergents; and higher olefins. The lower molecular weight olefins can be converted to sulfonates or alcohols by known commercial processes. The C₁₂-C₂₀ olefins find use in the detergent-products area. Lower molecular weight alcohols can be esterified with polyhydric acids, e.g., phthalic acid to form plasticizers for polyvinylchloride.

A broad range of both linear alpha- and internal-olefins may be produced by a two-stage process having an Alpha Olefins (AO) unit, which oligomerizes ethylene, and an Isomerization/Disproportionation (ID) unit, which converts alpha olefins to even and odd internal olefins. Excess light olefins in C₄-C₁₀ and heavy olefins in C₁₂₊ range produced in an oligomerization process may be converted to internal olefins in a desired range (currently C₁₁-C₁₄) by a sequential isomerization and disproportionation process. These detergent range (C₁₁-C₁₄) odd and even linear internal-olefins may be fed to an oxo-alcohol plant to produce C₁₂-C₁₅ semi-linear alcohols. Most of the alcohols are sold into the detergent markets with some of the alcohols being ethoxylated.

The product from the AO unit may be used in the production of linear low density polyethylene (LLDPE), synthetic engine (motor) oil, and in drilling mud formulations. However, the product from the AO unit may also be used as a feed to the ID unit which may include feed purification, olefin isomerization, olefin disproportionation, and product distillation.

During the isomerization process, the catalyst in the isomerization reactor becomes spent, the reactor catalyst may be replaced with new catalyst to return the process to full reactivity.

### SUMMARY

For use in an isomerization reactor, an isomerization catalyst composition includes an alumina based catalyst, wherein the alumina based catalyst has a pore volume in pores of less than 70 Å pore diameter of less than about 5% of Total Pore Volume, a pore volume in pores of greater than 350 Å pore diameter of less than 10% of Total Pore Volume, a median pore diameter by volume of less than 200 Å, a water pore volume of less than 1.2 cc/g and a surface area of greater than 130 m2/g. The isomerization catalyst composition may include Group I cations, Group II cations and mixtures thereof.

### BRIEF DESCRIPTION OF THE DRAWINGS

FIG. 1 is a simplified block flow diagram of an embodiment of a higher olefins process.

FIG. 2 is a simplified block flow diagram of an embodiment of an isomerization/disproportionation process.

### DETAILED DESCRIPTION

Embodiments described herein include a process for the oligomerization of ethylene into alpha olefins and a process for the production of internal olefins. The basic steps of the process include oligomerization, olefin isomerization and olefin disproportionation (metathesis).

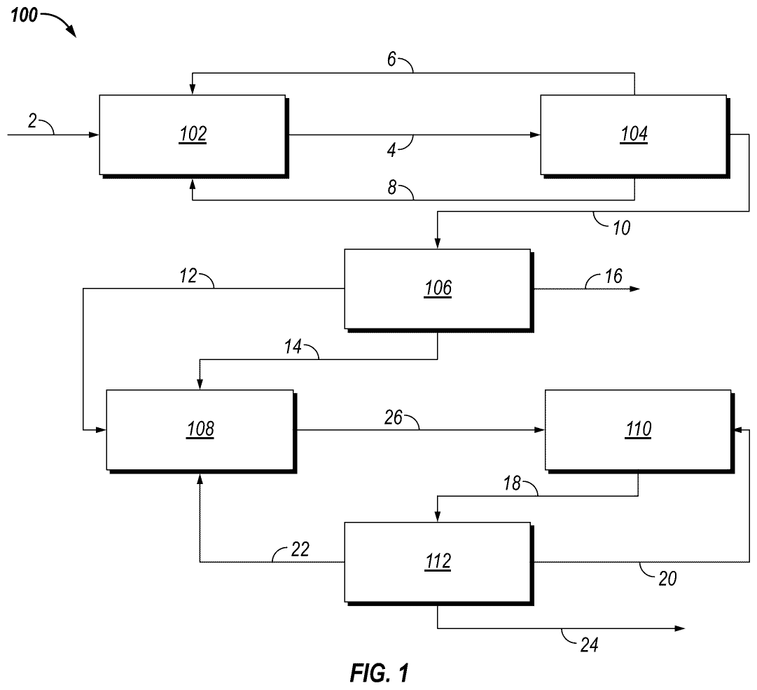

FIG. 1 shows a simplified block diagram of an oligomerization process. The oligomerization process 100 includes an oligomerization block 102, an olefinic phase separator block 104, a first distillation block 106, an isomerization block 108, a disproportionation block 110 and a second distillation block 112.

Ethylene 2 is fed to the oligomerization block 102. The ethylene is oligomerized in the presence of a non-Ziegler catalyst system (consisting of nickel and a complex ligand) to preferentially form a mixture of C₄-C₂₀₊ olefins, most of which are linear alpha-olefins. The reaction may be carried out in a polar solvent (typically 1,4-butanediol) at about 90-120° C. (194-248° F.) and 90-140 atm (1320-2050 psia). The oligomerization reactor is of vessel-type in which ethylene is bubbled in the catalyst-bearing solvent. The polar solvent is immiscible with the alpha olefins product, and contains catalyst materials in a dissolved state. Once formed, olefins can have little further reaction because most of them are no longer in contact with the catalyst. Oligomers formation generally follows the well-known Schultz-Flory equation.

The chain-growth catalyst is generically prepared by dissolving a nickel compound along with a bidentate ligand (e.g. diphenylphosphinobenzoic acid) and catalyst activator (sodium borohydride) in 1,4-butanediol. Nickel forms a complex compound with the ligand. After the oligomerization proceeds to the desired extent, the products 4 are withdrawn from the reactor and sent to the phase separator 104 where the solvent/catalyst 8 and unconverted ethylene 6 are separated from the oligomers and recycled to the oligomerization block 102. The oligomers product mixture 10, after undergoing a washing step for catalyst removal, is passed through the first distillation block 106, separating alpha olefins into the desired cuts. The oligomers product mixture 10 include a normal geometric distribution of ethylene oligomers, including high and low boiling range alpha-olefins.

The first distillation block 106 produces low boiling range alpha-olefins 12, high boiling range alpha-olefins 14, and alpha-olefin products 16. The low boiling range alpha-olefins 12 and high boiling range alpha-olefins 14 are sent to the isomerization block 108. The isomerization block 108 produces internal olefins 16 which are sent to the disproportionation block 110. The low boiling range alpha-olefins 12 and high boiling range alpha-olefins 14 are reacted over a heterogeneous catalyst to yield a range of the corresponding internal isomers. Unisomerized alpha olefins are kept to a minimum to reduce unwanted high molecular weight products and very short chain products in the downstream disproportionation block 110.

The disproportionation block 110 produces a product 18 including odd and even number internal olefins which is sent to the second distillation block 112. The disproportionation reaction occurs over a heterogeneous catalyst including cobalt, molybdenum, tungsten, or rhenium on alumina or silica substrates. The metathesis allows the low and high boiling range internal olefins to be disproportionated into a range of more useful molecular weight olefins. The disproportionation reactions are equilibrium controlled and conversions are a function of temperature and reactant concentrations.

The second distillation block 112 produces low boiling range internal-olefins 20, high boiling range internal-olefins 22, and internal-olefin products 24. The low boiling range internal-olefins 20 are recycled to the disproportionation block 110 and the high boiling range internal olefins 22 are recycled to the isomerization block 108.

In some embodiments, the mixture of low boiling range alpha-olefins 12 and high boiling range alpha-olefins 14 fed to the isomerization block 108 are a portion of the product from an oligomerization block 102 not destined for the alpha-olefin market.

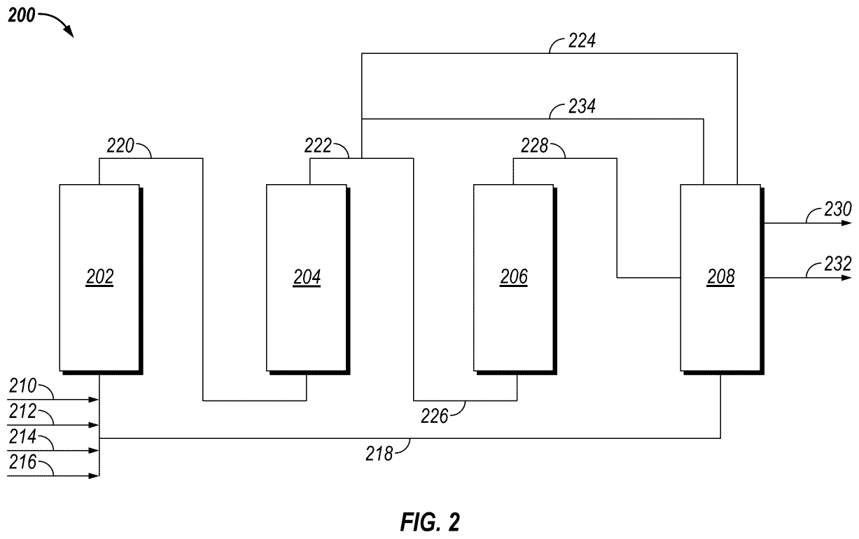

FIG. 2 is a block diagram of the isomerization/disproportionation process 200. In some embodiments, the isomerization/disproportionation process includes, but is not limited to, four major unit operations: a feed purification unit 202, an olefin isomerization unit 204, an olefin disproportionation unit 206 and a product distillation unit 208.

The isomerization/disproportionation process 200 randomizes the carbon chain lengths attached to a set of carbon-carbon double bonds. The randomization is accomplished via the olefin isomerization unit 204. The olefin isomerization reaction moves the location of the carbon-carbon double bond within the molecule, but does not change the number of moles passing through the reactor. Similarly, the disproportionation unit 206 fragments, then recombines, the olefins passing through the disproportionation unit 206, but does not change the number of moles. That is, the isomerization/disproportionation process 200 unit not only requires conservation of mass but also conservation of moles.

### Purification

The feed purification unit 202 removes catalyst poisons which may deactivate catalyst in either the olefin isomerization unit 204 or the olefin disproportionation unit 206. The feed to the feed purification unit 202 may include streams from the oligomerization block 102 including butene 210, light feed 212 (as shown in FIG. 1 as stream 12), and heavy feed 216 (as shown in FIG. 1 as stream 14). The catalyst poisons may include, but are not limited to, solvent degradation products (which may contain oxygen), water, peroxides, carbonyls, and ligand degradation products (most of which may contain phosphorus as a phosphine).

In some embodiments, the heavy feed 216 includes C₁₂₋₄₀₊ alpha olefins and the light feed 212 may include a mix of 1-hexene, 1-octene, and 1-decene. The mixed feed 214 may include olefins stripped from various fixed-bed operations as well as olefin washes used in those operations.

The butene 210, light feed 212, mixed feed 214, and heavy feed 216 may be combined with a heavy recycle 218 from the product distillation unit 208. The heavy recycle 218 may include the high molecular weight olefins not distilled overhead in the product distillation unit 208 (typically C₁₉₊).

The chemistry of the isomerization/disproportionation process 200 has the complexity that the average carbon number of the alpha olefin entering the isomerization/disproportionation process 200 must match the average carbon number of the isomerization/disproportionation product plus bleeds. A calculation, based on the flow rate and assumed carbon number of the isomerization/disproportionation products and bleeds, controls the alpha olefin feed rate and feed carbon number requirement. The calculation, using light feed 212 and mixed feed 214, and heavy recycle 218 as calculation inputs, calculates the required flows of the butene 210 and heavy feed 216 to meet the mass and carbon number requirement.

In some embodiments, the feed purification unit 202 may include one or more butene dryers, one or more purification beds and one or more light feed dryers or some combination thereof. The butene 210 may pass through the butene dryer containing an adsorbent such as, but not limited to, molecular sieve or alumina, typically 3A molecular sieve prior to mixing with the light feed 212, mixed feed stream 214, heavy feed 216 and heavy recycle 218. In other embodiments, the butene dryer may use any type of adsorbent, in particular molecular sieve. The adsorption capacity of the feed purification unit 202 may be controlled by the cycle times and age of the purification adsorbent in the feed purification unit 202. In this case, cycle time refers to the time between regenerations and age refers to the actual time the absorbent has resided in the equipment. The operational intention is to maintain the absorbent capacity at very high levels so that the isomerization/disproportionation process 200 conversion remains high and stable at all times. One skilled in the art would be able to determine the frequency and process to regenerate the feed purification unit 202 and also when to replace the adsorbents in the feed purification unit 202.

In some embodiments, the light feed 212 is sent to a light feed dryer prior to mixing with the butene 210, mixed feed 214, heavy feed 216, and heavy recycle 218. The light feed dryer may contain an adsorbent such as, but not limited to, molecular sieve or alumina, typically 4A molecular sieve.

The combined olefin stream of butene 210, light feed 212, mixed feed 214 and heavy feed 216 joins the heavy recycle 218 stream prior to entering the one or more purification (P) beds. The flow of the combined olefin stream flows from the bottom of the one or more purification beds to the top. The purification bed may be operated at temperatures ranging from about 110 to about 150° C., and in some embodiments from about 80 to about 140° C. and pressures ranging from about 1 to about 25 bar, and in some embodiment from about 3 to about 20 bar. In some embodiments, there are two purification beds to maintain the efficiency of the feed purification unit 202. The purification beds may be operated such that one purification bed is in-service while the other is undergoing regeneration. In other embodiments, there may be two purification bed operated in series with a third purification bed undergoing regeneration. In some embodiments, the heavy recycle 218 may be bypassed around the feed purification unit 202.

The one or more purification beds may contain one or more layers of adsorbent. The layers may have different compositions and one of skill in the art would be able to determine the amount of each adsorbent and the type of adsorbent for each layer.

In some embodiments, the adsorbent for the purification beds may be alumina shaped catalyst or molecular sieve, or may be alumina based catalyst or molecular sieve containing from about 5 wt % to about 20 wt % Group I or Group II cations or combinations thereof. The alumina may be alpha, beta, theta, or gamma alumina. The alumina based catalyst may contain in addition to alumina minor amounts of other inorganic oxides such as titania, zirconia, silica, phosphorous and the like. While the alumina material can contain small amounts of other components that do not materially affect the properties of the adsorbent. The alumina based catalyst, thus, can consist essentially of alumina. The phrase “consist essentially of” as used herein and in the claims with regard to the composition of the alumina based catalyst means that the alumina based material must contain the alumina and it may contain other components; provided such other components do not materially influence the adsorption properties of the final adsorbent composition.

The alumina based catalyst which may be employed in practice of this invention may be available commercially from catalyst suppliers or may be prepared by variety of processes.

Properties of the alumina based catalyst for the purification bed can be defined as follows:
Pore Volume in pores of less than 70 Å pore diameter: less than about 15% of Total Pore Volume, less than about 12% of Total Pore Volume, less than about 10% of Total Pore Volume;
Pore Volume in pores of greater than 350 Å pore diameter: less than about 10% of Total Pore Volume, less than 8% of Total Pore Volume, less than about 6% of Total Pore Volume;
Median pore diameter (MPD) correspond to the median pore diameter by volume: less than 120 Å, from about 80 to about 110 Å;
Water Pore volume: less than 1.10 cc/g, from about 0.65 to about 1.10 cc/g, from about 0.75 to about 1.0 cc/g.

Reference herein to the pore size distribution and pore volume of the alumina based catalyst are to those properties as determined by mercury penetration porosimetry. The measurement of the pore size distribution of the alumina based catalyst is by any suitable mercury porosimeter capable of working in the pressure range between atmospheric pressure and about 60,000 PSI, using a contact angle of 140 with a mercury surface tension of 474 dyne/cm at 25° C. Pore volume is defined as the total Volume using the mercury intrusion method as measured between atmospheric pressure and a pressure of about 60,000 psia.

Surface Area: from about 160 to about 400 m²/g, from about 200 to about 350 m²/g, from about 225 to about 325 m²/g. Surface area are measured by nitrogen adsorption, using the well-known B.E.T. method. The B.E.T. method of measuring Surface area has been described in detail by Brunauer, Emmet and Teller in J. Am. Chem. Soc. 60 (1938) 309-316, which is incorporated herein by reference.

In some embodiments, the alumina based catalyst includes Group I or Group II cations or combinations thereof. In some embodiments, the Group I cation is potassium or sodium and the Group I cations are selected from the group of potassium carbonate potassium hydroxide, potassium oxide, potassium acetate, potassium chloride, potassium sulfate, potassium nitrate, potassium acetylacetone, potassium citrate, potassium oxalate, and mixtures thereof, or equivalents of sodium salts. The amount of the metal may range from about 0 wt % to about 20 wt %, from about 7.5 wt % to about 15 wt %. In some embodiments, the metal may be incorporated into the alumina based catalyst by any suitable means or method known to those skilled in the art. For instance, the metal components can be co-mulled with the alumina of the alumina based catalyst during the formation of the agglomerate particles of the alumina based catalyst, or the metal components can be incorporated into the alumina based catalyst by impregnation, or the metal can be incorporated into the alumina based catalyst by a combination of methods. The metal components are incorporated into the alumina based catalyst in such amounts as to provide the concentration of metal components as described above. The alumina based catalyst with the incorporated metal components can be dried, or calcined, or both, in accordance with known methods to provide the final catalyst.

In some embodiments, the butene dryer, the light feed dryer and purification beds are regenerated on a timed basis which may be different for each unit. One skilled in the art will be able to determine when the butene dryer, the light feeds dryer and the purification beds should be regenerated. One skilled in the art would also know the regeneration procedure for the butene dryer and the light feed dryer would typically constitute a regeneration process of molecular sieves. Regeneration of the butene dryer and the light feed dryer involves vaporizing any residual hydrocarbon from the butene dryer and the light feed dryer. Regeneration of the purification bed involves burning of any residual hydrocarbon, purging oxygen and water after the burnoff, and cooling the purification bed in preparation for return to service.

The regeneration process for the purification beds may begin by displacing the butene 210, light feed 214, heavy feed 216 and heavy recycle 218 in the purification bed with either light feed 212 or an intermediate recycle, typically a C₄-C₁₁ stream from the product distillation unit 208. In other embodiments, the displacement fluid may be a C₁₅-C₁₅ stream from the product distillation unit 208. Next the bed is stripped with hot nitrogen (from about 260 to about 290° C.) to vaporize any recoverable hydrocarbon on the bed. A controlled burn of about 1-5% vol % oxygen in nitrogen is used to burn off residual hydrocarbon. Temperatures may get as high as about 450° C. in the bed. Next the bed is soaked in nitrogen at 450° C. and cooled in a nitrogen purge to below about 135° C. The bed may now be considered regenerated and ready to be put back online. One of ordinary skill in the art would be able to modify the regeneration procedure as necessary depending on the contents of the butene dryer, the light feed dryer and purification beds.

When the purification bed regeneration frequency is reduced, isomerization catalyst performance declines. With each regeneration, the adsorbent suffers a slight decline in surface area, which entails a concomitant decline in adsorption efficiency since adsorption efficiency relates directly to adsorbent surface area. Eventually, the surface area of the adsorbent sufficiently reduces that necessitates dumping the exhausted adsorbent and replacing it with new adsorbent. A given charge of purification bed adsorbent may fulfill its function for several years, such as four to seven years.

The discharge streams of the in-service purification bed and the light feed dryer combine and exit the feed purification unit 202 as a purified feed 220 which is fed to the isomerization unit 204.

### Isomerization

The isomerization unit 204 may include multiple reactors operating in series. The isomerization unit 204 takes alpha olefin products and moves carbon-carbon double bonds from the alpha position to other positions along the carbon chain length and also achieves an equilibrium distribution of the carbon-carbon double bonds along the carbon chain lengths. Olefin isomerization creates a mixture of carbon chain lengths attached to the carbon-carbon double bonds. The olefin exiting the isomerization unit 204 is isomerization product 222.

In some embodiments, the isomerization unit 204 includes four fixed bed isomerization reactors where three reactors are operated in series, parallel or a combination at any one time with the fourth reactor being in turnaround. In some embodiments, the isomerization reactor having the oldest (longest catalyst on-cycle) catalyst charge is placed via rotation in the first isomerization reactor position. The next oldest catalyst charge is placed in the second isomerization reactor position and the youngest catalyst charge is placed in the third isomerization reactor position. When a newly turned around isomerization reactor is brought online, the positions of the isomerization reactors are adjusted accordingly. In some embodiments, the isomerization reactors operate in a downflow direction.

In some embodiments, the reactor having the oldest isomerization catalyst charge is placed via rotation in the first isomerization reactor position, i.e., receiving the purified feed 220. The purified feed 220 may include heavy recycle 218, which is at least fifty percent isomerized. The use of multiple isomerization reactors in series will bring the olefin mixture to 100% isomerization.

The one or more isomerization reactors may contain one or more layers of isomerization catalyst. The layers may have different compositions and one of skill in the art would be able to determine the amount of each catalyst and the type of catalyst for each layer. In some embodiments, a guard bed layer of alumina may be placed above and below the isomerization catalyst. The guard bed layer of alumina may be one or more layers of from about 10 to 20% of the total amount of isomerization catalyst. The guard bed layer may include varying pore sizes of alumina. In some embodiments, the guard bed may have a median pore diameter by volume ranging from about 115 to about 200 Å.

In some embodiments, the isomerization catalyst for the isomerization reactor may be an alumina based catalyst or molecular sieve. The alumina may be alpha, beta, theta, or gamma alumina. The alumina based catalyst may contain in addition to alumina minor amounts of other inorganic oxides such as titania, zirconia, silica, phosphorous and the like. While the alumina based catalyst can contain small amounts of other components that do not materially affect the properties of the catalyst, the alumina based catalyst should generally consist essentially of alumina. The phrase “consist essentially of” as used herein and in the claims with regard to the composition of the alumina based catalyst means that the alumina based catalyst must contain the alumina and it may contain other components; provided such other components do not materially influence the catalytic properties of the final catalyst composition.

The alumina based catalyst may be available commercially from catalyst suppliers or may be prepared by variety of processes.

Properties of the alumina based catalyst for the isomerization catalyst can be defined as follows using the test methods descried above:
Pore Volume in pores of less than 70 Å pore diameter: less than about 5% of Total Pore Volume, from about 5% to about 0.01% of Total Pore Volume, from about 3% to about 0.01% of Total Pore Volume;
Pore Volume in pores of greater than 350 Å pore diameter: less than 10% of Total Pore Volume, less than about 8% of Total Pore Volume, less than about 6% of Total Pore Volume;
Median pore diameter (MPD) corresponds to the median pore diameter by volume: less than 200 Å, from about 90 to about 180 Å, or from about 105 to about 165 Å;
Water Pore volume: less than 1.20 cc/g, from about 0.79 to about 1.10 cc/g;
Surface Area: from about 100 to about 400 m²/g, from about 150 to about 350 m²/g, from about 170 to about 300 m²/g; and
Pore diameter range of the most abundant 66% pore from Hg PSD method of 60 to 6K (PSD Width 66% (60 k-6 k)): from about 10 to about 100 Å.

In some embodiments, the alumina based catalyst may be contain Group I or Group II cations or combinations thereof. In some embodiments, the Group I cation is potassium or sodium and the Group I cations are selected from the group of potassium carbonate potassium hydroxide, potassium oxide, potassium acetate, potassium chloride, potassium sulfate, potassium nitrate, potassium acetylacetone, potassium citrate, potassium oxalate, and mixtures thereof, or the equivalents of sodium salts. The amount of the metal may range from about 0 wt % to about 20 wt %, from about 0.5 wt % to about 15 wt %. In some embodiments, the metal may be incorporated into the alumina based catalyst by any suitable means or method known to those skilled in the art. For instance, the metal components can be co-mulled with the alumina of the alumina based catalyst during the formation of the agglomerate particles of the alumina based catalyst, or the metal components can be incorporated into the alumina based catalyst by impregnation, or the metal can be incorporated into the alumina based catalyst by a combination of methods. The metal components are incorporated into the alumina based catalyst in such amounts as to provide the concentration of metal components as described above. The alumina based catalyst with the incorporated metal components can be dried, or calcined, or both, in accordance with known methods to provide the isomerization catalyst.

In some embodiments, the alumina based catalyst or the cation impregnated isomerization catalyst is further impregnated with from about 0.5 to about 15 wt % of a metal of Group I metal, Group II metal or combinations thereof. In some embodiments, the metal may be impregnated as a liquid or a eutectic alloy. The eutectic alloy may be a sodium potassium eutectic. In one embodiment, the process of impregnating the alumina based catalyst or isomerization catalyst may include the following steps. First, the alumina based catalyst or isomerization catalyst is heated in a vessel to between 700° F. and 900° F. for about 12 to 18 hours. Next, the alumina based catalyst or isomerization catalyst is then transferred to a mixing vessel. In the mixing vessel, once the alumina based catalyst or isomerization catalyst temperature has cooled to about 500° F., NaK is impregnated onto the alumina based catalyst or isomerization catalyst. In some embodiments, the combination of the 5 weight % potassium on the isomerization catalyst with the additional 5 weight % NaK results in about 70% longer (from 14 to 24 days) catalyst run time than previous versions of isomerization catalyst which had only NaK.

During the impregnation and mixing phase, the mixing vessel is kept as close to isothermal as possible at a desired setpoint ranging from 400 to 500° F. for 4 to 6 hours of impregnation time. The temperature is controlled by using hot oil flowing through the mixing vessel jacket. The production of isomerization catalyst under isothermal conditions results in an additional increase in run time of 80% (from 24 to 43 days). While described for impregnating a NaK euctectic alloy, the same procedure may be used for impregnating the alumina based catalyst or the cation impregnated isomerization catalyst with other metals or metal alloys.

During the isomerization process, the catalyst of the isomerization unit 204 is deactivated. The isomerization catalyst contains a highly reactive (pyrophoric) metal, meaning a turnaround procedure is performed which ensures safe disposal. The turnaround prepares the catalyst to be dumped from one of the isomerization reactors. The turnaround procedure may include the following steps: hydrocarbon washing of the catalyst; deactivating the catalyst; water washing the catalyst; and dumping the catalyst.

Hydrocarbon washing: Once an isomerization reactor is ready to come offline for turnaround, the full range olefins (C₄-C₄₀₊) are displaced (hydrocarbon wash) with hot olefin (C₄-C₁₁ or C₁₅-C₁₈). In some embodiments, the feed is stopped to the reactor which is to be taken offline. The hot olefin wash enters the top of the reactor and washes the reactor by going through the reactor and out the bottom, returning to the process piping. The hot olefin wash clears any water out of the header and heats the header to the hot olefin wash temperature, about 121° C. The hot olefin wash is flushed through the isomerization reactor for about 2.5 hours. Once the hydrocarbon wash is completed, the offline isomerization reactor is isolated from the process.

Catalyst deactivation: Once the offline isomerization reactor has been hot olefin washed, the hot olefin left in the isomerization reactor and the olefin inventoried in the Deactivation HC Storage Vessel are cooled down. The deactivating manifold is lined up in upflow so that the olefin will flow from the Deactivation HC Storage Vessel into the bottom of the isomerization reactor, out the top of the I Reactor and back into the Deactivation HC Storage Vessel. A circulation flow through the I Reactor is established which also goes through a cooling water exchange to cool the reactor and the Deactivation HC Storage Vessel. Once the reactor and the Deactivation HC Storage Vessel contents are cooled, to a maximum of about 100° F., the system is ready for the deactivation. Once cooled, a small amount of water is injected into the circulation flow. This slowly deactivates the NaK as the water level slowly builds in the reactor and eventually spills over into the Deactivation HC Storage vessel. This will mark the end of the deactivation phase.

Water Wash: The water wash step provides another opportunity for water to contact any pyrophoric material in the offline I Reactor and flush the NaOH and KOH out of the isomerization reactor. The Water Pump is then started and the wash water flow valve is opened completely to flush through the deactivation header for about 15 minutes. The deactivation header bypass valve is then closed and the wash water flow is lined up through the bottom of the I Reactor, out the top, and into the deactivation HC Surge Vessel. Once the level in the deactivation HC Surge Vessel reaches a prescribed level, the wash water flow and Water Pump are stopped. A prescribed level of water is drained from the bottom of the I Reactor into the deactivation HC Surge Vessel to create a vapor space in the I Reactor. Sparge nitrogen is then lined up to the bottom of the I Reactor, and the associated flow control valve is opened wide to sparge nitrogen in the bottom of the I Reactor, out the top of the reactor, and into the deactivation HC Surge Vessel where it vents to the vent header system. This sparge continues for an hour to help purge any hydrocarbon vapors. After an hour, the 1st nitrogen sparge is stopped and the deactivation HC Surge Vessel level controller is lined up to allow water drainage from the bottom boot at a prescribed level. The Water Pump is restarted and the wash water is flowed for approximately six hours. The wash water will flow through the I Reactor bottom and into the deactivation HC Surge Vessel where it will be drained to the sewer through the Solids Collection Sump. Once the target totalized flow is attained, the water pump will be stopped. The deactivation HC Surge Vessel level control valve will close and, after a timed period, an isolation valve will also close to ensure positive isolation.

These steps may be repeated for a 2nd nitrogen sparge and 2nd water wash, a 3rd nitrogen sparge and 3rd water wash, and 4th nitrogen sparge and 4th water wash.

Drying/dump: The isolation valve is closed to keep the water in the I reactor. The Water Pump is stopped and the I Reactor pressure is increased. Upon opening the bottom dump valve, the catalyst and water is dumped into a catalyst trailer. The I reactor (and associated parts) is then sparged with nitrogen and washed with water. Next, the I reactor is dried in preparation for loading of fresh catalyst.

### Disproportionation

The isomerization product 222 combines with the light recycle 234 and the intermediate recycle 224 to form disproportionation feed 226. The disproportionation feed 226 enters the disproportionation unit 206 and a disproportionation product 228 is produced. The disproportionation (metathesis) unit 206 allows the low and high boiling range internal olefins to be disproportionated into a range of more useful molecular weight olefins with the total number of molecules remaining essentially unaltered.

Olefin metathesis involves breaking double bonds on different olefin molecules, then recombining the fragments, for example:

5R1CH═CHR2+5R3CH═CHR4↔R1CH═CHR3+R1CH═CHR4+R2CH═CHR4+R2CH═CHR3R1CH═CHR1+R2CH═CHR2+R4CH═CHR4+R3CH═CHR3+R1CH═CHR2+R3CH═CHR4

The resulting product is a statistical distribution of the “arms” appended to the original double bonds.

For example, a C₄ olefin and a C₂₄ olefin can be disproportionated into two C₁₄ olefins. Olefin disproportionation reactions are equilibrium controlled and conversions are a function of temperature and reactant concentrations.

The disproportionation may be carried out either batchwise or continuously using a fixed catalyst bed or a fluidized catalyst bed or any other mobile catalyst contacting process. Preferred reaction conditions of the disproportionation process e.g. temperature, pressure flow rates etc. may vary somewhat depending upon the specific catalyst composition, the particular feed olefin, desired products etc. The process is typically carried out at temperatures in the range of from 150 to 400° C. and under a pressure in the range of from 1 to 50 bara. More preferably a temperature in the range of from 200 to 300° C. is used.

The operable range of contact time for the disproportionation reaction depends primarily upon the operating temperature and the activity of the catalyst, which is influenced by surface area, promoter concentration, activation temperature, etc. In general, the distribution of products is not drastically altered by variation in contact time. Shorter contact times are usually associated with higher temperatures, but, when larger amounts of higher molecular weight products are desired, a suitable combination of contact time and temperature is selected. With proper selection of conditions and contact times, very high efficiency of conversion to desired products can be obtained.

The disproportionation unit 206 may include multiple disproportionation reactors. The number of disproportionation reactors may be determined by one skilled in the art. In some embodiments, the disproportionation unit 206 includes five disproportionation reactors, four disproportionation reactors operating in parallel while the fifth disproportionation reactor is being regenerated. When a disproportionation reactor completes regeneration, it is placed on-line, and the disproportionation reactor that has been on-line the longest goes into regeneration. In other embodiments, multiple reactors may also be placed in series. The multiple reactors may not be uniformly sized.

In some embodiments, the volumetric olefin flowrate through a given disproportionation reactor depends upon the time the disproportionation reactor has been online since the last regeneration i.e., newly regenerated disproportionation catalyst receives a proportionately higher volumetric olefin flowrate than those disproportionation catalysts with longer online operating times. In other embodiments, disproportionation feed is distributed evenly to the online disproportionation reactors.

The reactors in the disproportionation unit 206 are filled with a disproportionation catalyst. Embodiments of disproportionation catalysts may be found in in U.S. Pat. Nos. 4,754,099, 4,962,263, 4,996,386, 4,956,516 and European Patent No. 0319065.

In some embodiments, the disproportionation catalyst may comprise a molybdenum catalyst with an alumina based catalyst.

Properties of the disproportionation catalyst can be defined as follows:
Pore Volume in pores of less than 350 Å pore diameter: less than about 10% of Total Pore Volume, less than 8% of Total Pore Volume, less than about 6% of Total Pore Volume;
Median pore diameter (MPD) corresponds to the median pore diameter by volume: from about 55 to about 95 Å, from about 60 to about 80 Å;
Water Pore volume: greater than about 0.5 cc/g, to less than about 1.0, from about 0.55 to about 0.85 cc/g, from about 0.60 to about 0.82 cc/g;
Surface Area: greater than about 200 m²/g, greater than 240 m²/g, greater than 260 m²/g.

In some embodiments, the disproportionation catalyst may also include less than about 12 wt % of a Group 6 metals and from about 0 to about 10 wt % of a Group 14 metal. The Group 6 metals may be selected from the group consisting of molybdenum, tungsten and mixtures thereof. In some embodiments, the amount of molybdenum is less than about 12 wt %, ranges from about 2 to about 10 wt %, or from about 4 to about 8 wt %. The Group 14 metal may be silicon. In some embodiments, the disproportionation catalyst does not contain silicon. In other embodiments, the disproportionation catalyst may include from about 1 to about 5 wt % silicon.

The disproportionation catalysts may be generally prepared according to conventional methods such as impregnation, wherein a carrier is impregnated with a solution of metals, co-precipitation, wherein a carrier compound and metals are simultaneously precipitated, or co-mulling, wherein dry powders are mixed with a suitable extrusion aid such as water and extruded, or combinations thereof.

In some embodiments, the metal and/or silicon may be incorporated into the alumina based catalyst by any suitable means or method known to those skilled in the art. For instance, the metal and/or silicon components can be co-mulled with the alumina of the alumina based catalyst during the formation of the agglomerate particles of the alumina based catalyst, or the metal and/or silicon components can be incorporated into the alumina based catalyst by impregnation, or the metal and/or silicon can be incorporated into the alumina based catalyst by a combination of methods. The metal and/or silicon components are incorporated into the alumina based catalyst in such amounts as to provide the concentration of metal and/or silicon components as described above. The alumina based catalyst with the incorporated metal components can be dried, or calcined, or both, in accordance with known methods to provide the disproportionation catalyst.

Since disproportionation shifts the double bond towards one end of the resulting olefin, the disproportionation product 228 is not at equilibrium with respect to double bond distribution. Because alpha olefins disproportionate into lower molecular weight olefins; i.e., “lights”, the alpha olefin present in disproportionation feed 226 should be minimized, otherwise excessive amounts of “lights” (ethylene and propylene) may be formed. The ethylene and propylene may polymerize on the disproportionation catalyst, thereby inducing coke formation on it which reduces its reactivity.

Disproportionation catalysts may be activated multiple times to extend service life. In some embodiments, a possible activation process includes the following steps: a) a hot wash (temperature up to about 250° F. (about 120° C.)) using the intermediate recycle consisting of C₄ to C₁₀ internal olefins is performed by flowing through the catalyst bed to remove the bulk of the heavy hydrocarbon and/or coke; b) the hot recycle stream is then removed from the catalyst bed using nitrogen pressure; c) a stripping step is then employed which exposes the catalyst bed to hot nitrogen (temperatures up to about 550° F. (288° C.)) to vaporize residual hydrocarbon and water; d) residual hydrocarbon and coke is removed via combustion by increasing the temperature (up to about 750 to 850° F. (400 to 455° C.)) and oxygen concentration (from about 0.4 to about 0.8, up to 2% by feeding air); and e) stopping oxygen flow resulting in hot nitrogen circulating in the bed. In some embodiments, an external drier utilizing mol sieves may be used to dry the nitrogen, removing the water formed during combustion. The external drier ensures moisture is removed from the catalyst bed prior to placing back into service. In some embodiments, higher temperatures may be employed depending on the nature of the coking.

To minimize ethylene and propylene accumulation in the ID unit these “lights” may be vented. The alpha olefins and dimerized branched olefin are relatively unreactive in the disproportionation unit 206 and accumulate in the heavy recycle stream which lowers the reactivity of the heavy recycle stream. To maintain reasonable reactivity, these unreactive olefins may be purged from the distillation unit 208.

From the disproportionation unit 206, the disproportionation product 228 (which is combined from the multiple reactors) is sent to the product distillation unit 208. In some embodiments, the product distillation unit includes a number of distillation columns for providing a slate of olefin products along with recycle to either the disproportionation unit 206 or the feed purification unit 202. In some embodiments, recycle may also be sent to the isomerization unit 204. The light recycle 234 and the intermediate recycle 224 may be recycled to the disproportionation unit 206 and the heavy recycle 218 may be recycled to the feed purification unit 202.

In some embodiments, the product distillation unit 208 produces C11-12 olefins 230 and C15-16 olefins 232. In other embodiments, the product distillation unit 208 may be designed by one skilled in the art to also produce products including, but not limited to, C80 olefins, a mixture of C8,9,10 internal olefins; C10 olefins; C15-16 olefins; C15-18 olefins, a mixture of C15,16,17,18 internal olefins.

In some embodiments, there may be additional internal recycle streams from within the olefin disproportionation unit 206. These recycles may include light recycle (primarily C4 and C5 olefins but may include up to C10 olefins) and intermediate recycle (C4-10 internal olefins).

Although a few embodiments of the disclosure have been described in detail above, those of ordinary skill in the art will readily appreciate that many modifications are possible without materially departing from the teachings of this disclosure.

Accordingly, such modifications are intended to be included within the scope of this disclosure as defined in the claims.

1. A composition comprising an alumina based catalyst, wherein the alumina based catalyst has a pore volume in pores of less than 70 Å pore diameter of less than about 5% of Total Pore Volume, a pore volume in pores of greater than 350 Å pore diameter of less than 10% of Total Pore Volume, a median pore diameter by volume of less than 200 Å, a water pore volume of less than 1.2 cc/g and a surface area of greater than 130 m²/g.

2. The composition of claim 1, wherein the alumina based catalyst further comprises from about 0 wt % to about 20 wt % Group I or Group II cations or combinations thereof.

3. The composition of claim 2 having a pore diameter range of the most abundant 66% pore from Hg PSD method of 60 to 6K ranging from about 10 to about 100 Å.

4. The composition of claim 2, wherein the alumina support further comprises Group I cations.

5. The composition of claim 3, wherein the Group I cations are selected from the group of potassium carbonate potassium hydroxide, potassium oxide, potassium acetate, potassium chloride, potassium sulfate, potassium nitrate, potassium acetylacetone, potassium citrate, potassium oxalate, and mixtures thereof, or the equivalents of the sodium salts.

6. The composition of claim 1 for use as a catalyst.

7. The composition of claim 1, further comprising from about 0.5 to about 15 wt % of a metal comprising Group I metals, Group II metals or combinations thereof.

8. The composition of claim 7, wherein the metal comprises a sodium potassium alloy.

9. A method of producing a catalyst comprising the steps of:
a) heating the catalyst of claim 2 to a temperature ranging from about 700° F. to about 900° F. for about 12 to about 18 hours to provide a heated catalyst;
b) cooling the heated catalyst from step a) to a temperature of from about 400 to about 500° F. to produce a cooled catalyst; and
c) mixing the cooled catalyst with a liquid metal comprising from about 0.5 to about 15 wt % of Group I metals, Group II metals or combinations thereof.

10. The method of claim 9, wherein the mixing is done at isothermal conditions.

11. The method of claim 9, wherein the mixing is done for a time ranging from about 4 to about 6 hours.
